(19) United States
(12) Patent Application Publication
(10) Pub. No.: US 2021/0121139 A1
(43) Pub. Date: Apr. 29, 2021

(54) PRESSURE TOUCH SENSITIVE PATIENT TABLE FOR TOMOGRAPHIC IMAGING

(71) Applicant: KONINKLIJKE PHILIPS N.V., EINDHOVEN (NL)

(72) Inventors: Andriy ANDREYEV, WILLOUGHBY HILLS, OH (US); Chuanyong BAI, SOLON, OH (US)

(21) Appl. No.: 16/605,881

(22) PCT Filed: Apr. 18, 2018
(86) PCT No.: PCT/EP2018/059813

§ 371 Date: Oct. 17, 2019

Related U.S. Application Data
(60) Provisional application No. 62/488,196, filed on Apr. 21, 2017.

(51) Int. Cl. A61B 6/04; A61B 6/00

(52) U.S. Cl. CPC A61B 6/0407; A61B 6/5276; A61B 2562/046; A61B 2562/0247; A61B 6/541

(57) ABSTRACT

A device (10) for a patient to lie on during a medical imaging procedure includes a main body (12). A matrix of pressure sensors (16) disposed on a top surface (14) of the main body are configured to continuously measure pressure across the top surface. At least one electronic processor (22) is operatively connected to read the pressure sensors. A non-transitory storage medium stores instructions readable and executable by the at least one electronic processor to use the matrix of pressure sensors to perform at least one of: a sag estimation operation (200); a motion estimation operation (300); and a respiratory monitoring operation (400).

### FIELD

The following relates generally to the medical imaging arts, image positioning arts, image motion correction arts, and related arts.

### BACKGROUND

Real time motion detection and accurate patient positioning tracking is an important interest in medical imaging and one of the keys to precision medicine. Some progress has been made by using real time video tracking devices. However, these devices and techniques require expensive high resolution and depth sensing optics and electronics, precise aiming, and complex and computation heavy processing of the acquired videos.

Additionally, the tracking of breathing patterns allows for the correction of respiratory motion or respiratory gating during patient scans (such as in computed tomography (CT) and positron emission tomography (PET) scans). Simple but reliable detection and tracking of the respiratory motion can significantly improve image quality and quantitation by using the tracking information in data acquisition and processing. Conventional approaches use different optical devices, or pressure sensors in bellows, using ECG leads for cardiac beating and respiratory motion detection, etc.

The following discloses new and improved systems and methods to overcome these problems.

### SUMMARY

In one disclosed aspect, a device for a patient to lie on during a medical imaging procedure includes a main body. A matrix of pressure sensors disposed on a top surface of the main body are configured to measure pressure across the top surface. At least one electronic processor is operatively connected to read the pressure sensors. A non-transitory storage medium stores instructions readable and executable by the at least one electronic processor to use the matrix of pressure sensors to perform at least one of: a sag estimation operation; a motion estimation operation; and a respiratory monitoring operation.

In another disclosed aspect, a device for a patient to lie on during a medical imaging procedure includes an imaging device. A main body is arranged to load a patient into the imaging device for imaging. A matrix of pressure sensors disposed on a top surface of the patient support are configured to measure pressure across the top surface. At least one electronic processor is operatively connected to read the pressure sensors. A non-transitory storage medium stores instructions readable and executable by the at least one electronic processor to use the matrix of pressure sensors to perform at least one of: a sag estimation operation; a motion estimation operation; and a respiratory monitoring operation.

In another disclosed aspect, a method of monitoring a patient during an image acquisition procedure includes: reading pressure sensors that contact a portion of the patient's body on a top surface of a main body to obtain pressure data; and based on the obtained pressure data, estimating a sag of the main body.

One advantage resides in providing a system to provide accurate estimation of position and movement of a patient undergoing imaging.

Another advantage resides in providing context-sensitive remedial action in response to a detected movement of a patient undergoing imaging.

Another advantage resides in tracking respiration information without attaching an additional device to a patient and which is applicable to monitoring respiration of a patient in either a prone (i.e. face-down) or supine (i.e. face-up) position.

Another advantage resides in accurately determining the amount of table sag in real time.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the invention.

FIG. 1 diagrammatically illustrates a device for a patient to lie on during a medical procedure in accordance with one embodiment.

FIG. 2 diagrammatically shows an operational flow chart for one example operation of the device of FIG. 1.

FIG. 3 diagrammatically shows an operational flow chart for another example operation of the device of FIG. 1.

FIG. 4 diagrammatically shows an operational flow chart for another example operation of the device of FIG. 1.

### DETAILED DESCRIPTION

The following discloses various embodiments which leverage an array of pressure sensors disposed on a patient table to address important issues in the field of medical imaging. In some illustrative embodiments, the pressure sensors are used to detect the identity of a body part which is moved by the patient (e.g., a leg or arm), the time of the movement, and in some embodiments the direction of the movement. This information provides guidance on whether there is a need to redo the scan, or apply motion correction to certain parts of the data.

Respiratory information can also be tracked based on the pressure readings, without the need of any additional device to be attached to the patient. In some embodiments, a pressure magnitude versus time signal is measured, from which the respiratory cycle can be estimated. Advantageously, this approach is operative even in the case of a supine patient for when the chest rises away from the table during inhalation. As recognized herein the expansion of the chest volume during respiratory cycle produces a body mass redistribution executing downward force on the patient table whose magnitude can be measured by the pressure sensors. This pressure magnitude is expected to vary with the extent and direction of chest expansion and contraction, so that the pressure magnitude versus time signal is expected to vary in correlation with the respiratory cycle. It is similarly contemplated to monitor cardiac cycling via (the higher frequency component of) the pressure magnitude versus time signal.

In some embodiments, the pressure sensor readings are used to more accurately assess table sag. Sag occurs when the patient support (e.g. table, pallet, or other main body supporting the patient) is positioned in a cantilevered position. For example, in a hybrid PET/CT or SPECT/CT imaging system, the patient support generally includes a couch or the like having a tabletop (or pallet, or otherwise named main body) that is moved into the CT gantry and (if movement continues) into the PET or SPECT gantry. In such a design, the tabletop or pallet may be cantilevered, with the end that projects into the CT or PET/SPECT gantry is not supported. This unsupported end can sag downward under the weight of the patient. The sag depends on the stiffness of the tabletop or pallet, and is conventionally recognized to further depend on the weight of the patient supported by the tabletop or pallet. However, as recognized herein, the sag is more specifically dependent on the weight distribution being supported by the tabletop or pallet. Thus, in embodiments of sag estimation disclosed herein, the array of pressure sensors enables determination of the distribution of weight over the patient table—from this weight distribution, the sag may be more accurately estimated. In one approach, the center of mass (COM) and total weight of the patient is used to more accurately estimate the table sag, versus estimation based on patient weight alone. In another approach, the combined effect of the sag contributions of the portions of the weight distribution are computed, e.g. by integration or summation, to estimate the table sag. Using the weight distribution, rather than the patient weight, provides more accurate position dependent table sag estimation. The table sag is also measured in real time, which is advantageous as the patient table typically bends due to patient weight by an increasing amount as the patient table is extended further into the gantry for scanning (e.g. producing an increasingly long cantilevered table length). By accurately measuring table sag in real time, the required correction coefficients for proper PET/CT images realignment can be derived.

These approaches leverage a pressure touch sensitive layer disposed on the top of the patient table. The pressure sensitive layer can be constructed of a grid of individual pressure sensitive cells or elements. The array of pressure sensors cover at least that portion of the surface area of the top of the patient table which may be credibly expected to come in contact with the patient. An electronic processor is operatively connected to read the pressure sensors and to interpret the information from the sensors and compute the real-time patient weight distribution and other information, e.g. patient contour for the portion of the patient touching the sensor array, passing it further to the image reconstruction chain. The array of pressure sensors can be formed integrally with the top of the patient table (e.g. embedded into the top surface of the patient table), or the pressure sensors can be attached separately to a table cover or fitted sheet that is then disposed over a patient table surface for the same purpose, which is advantageous to enable retrofitting an existing patient table without having to completely redesign/replace already released couch models.

For motion assessment, the sensors can be used to detect when a movement occurs, what body part moves (based on the patient's footprint and expected anatomy), and the direction and magnitude of movement. For example, the sensors can detect the patient moved the left leg to the right. This information can be variously used. In the case of PET/CT, the movement of any body part that has already been imaged by both PET and CT is not problematic. If the moved body part has not yet been imaged, then various remedial actions can be taken. If the movement occurs during imaging of the moved body part, then the imaging data sets acquired before/after the motion are each separately reconstructed, and optionally later merged by spatial registration. If the movement occurs early in imaging of the body part, the early data may be discarded, and optionally the imaging time can be extended to compensate the discarded early portion. If the movement occurs before PET imaging of the moved body part commences but after CT imaging of the moved body part, then it is contemplated to ask the patient to move the body part back to its original position. In making this “correction”, the pressure sensors can be used to detect when the body part is back in its original position.

Respiratory monitoring using the pressure sensors is based on the insight that even if the patient is lying on the back (supine position), the respiration produces modulation of the magnitude of pressure applied to the table. Thus, respiratory cycle can be extracted from the pressure magnitude versus time curve acquired by pressure sensors contacting the backside of the supine patient. Cardiac cycling monitoring is also contemplated by this technique.

Table sag correction uses the pressure sensors to measure the weight distribution over the table, so as to provide a more accurate sag estimation as compared with estimates that are based on the patient's total weight. Various approaches can be employed. In one approach, the center of mass (COM) and total weight are determined from the pressure sensor measurements, and this is used in an empirical look-up table or by applying a first principles beam deflection equation to determine the table sag. In a more precise approach, a look-up table or beam deflection equation is applied on a per-element basis, for each weight component measured by each pressure sensor (or by contiguous groups of pressure sensors) and the total sag is then the sum of these “regional” sag contributions. Advantageously, since the pressure sensors monitor the weight distribution in real-time, changes in sag due to patient movement or repositioning during the imaging session are made feasible.

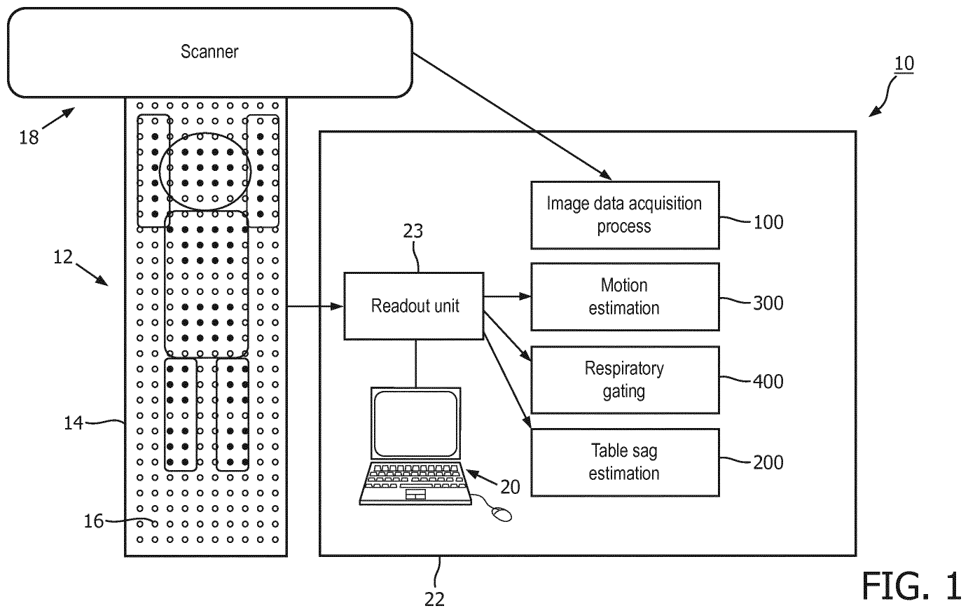

With reference to FIG. 1, an illustrative device 10 for a patient to lie on during a medical imaging procedure is shown. As shown in FIG. 1, the device 10 includes a main body 12. In one example, the main body 12 can comprise a table for the patient to lie on. In another example, the main body 12 can comprises a top, padded portion of a table (i.e., without any table legs). In other examples, the main body 12 can comprises a bench or a couch for the patient to lie on. The main body 12 includes a top surface 14 on which a patient lies for an imaging procedure.

A matrix of pressure sensors 16 are disposed on the top surface 14 of the main body 12. As shown in FIG. 1, the pressure sensors 16 are distributed across the length and width of the top surface 14; although the pressure sensors can be disposed on only a portion of the top surface. The pressure sensors 16 are configured to continuously measure pressure across the top surface 14. For example, the pressure sensors 16 can measure pressure values when a patient lies on the top surface 14. The pressure sensors 16 measure pressure readings at the location of the different parts of the patient's body that overlie the sensors. The pressure sensors 16 can employ substantially any type of pressure sensing technology, e.g. they may be piezoresistive strain sensors, capacitive pressure sensors in which pressure compressively reduces the dielectric thickness of a capacitor, electromagnetic sensors in which pressure-induced displacement of a diaphragm or other movable element is detected as an inductive change or the like, a piezoelectric sensor, or so forth.

In some examples, the device 10 can also include or operate with an imaging device 18, such as a hybrid positron emission tomography (PET)/computer tomography (CT) scanner configured to obtain images of a patient when the patient lies on the top surface 14 of the main body 12. However, it will be appreciated that the imaging device 18 may more generally be any suitable imaging modality scanner (e.g., magnetic resonance, a gamma camera for single photon emission computed tomography, X-ray, and the like). A computer 20 or other electronic device including an electronic processor 22 is in electrical communication with the pressure sensors 16. The computer 20 that includes the at least one electronic processor 22 which includes or is operatively connected with a pressure sensor readout unit 23 to read the pressure sensors 16. The at least one electronic processor 22 is operatively connected with a non-transitory storage medium that stores instructions which are readable and executable by the electronic processor 22 to perform disclosed operations including controlling the imaging device 18 to perform an imaging data acquisition process 100. Additionally, the non-transitory storage medium may store instructions readable and executable by the electronic processor 22 to perform one or more operations upon receiving pressure values from the pressure sensors 16, including for example at least one of (1) a sag estimation operation 200; (2) a motion estimation operation 300; and (3) a respiratory monitoring (and optional respiratory gating) operation 400, each of which is described in more detail below. The non-transitory storage medium may, for example, comprise a hard disk drive, RAID, or other magnetic storage medium; a solid state drive, flash drive, electronically erasable read-only memory (EEROM) or other electronic memory; an optical disk or other optical storage; various combinations thereof; or so forth.

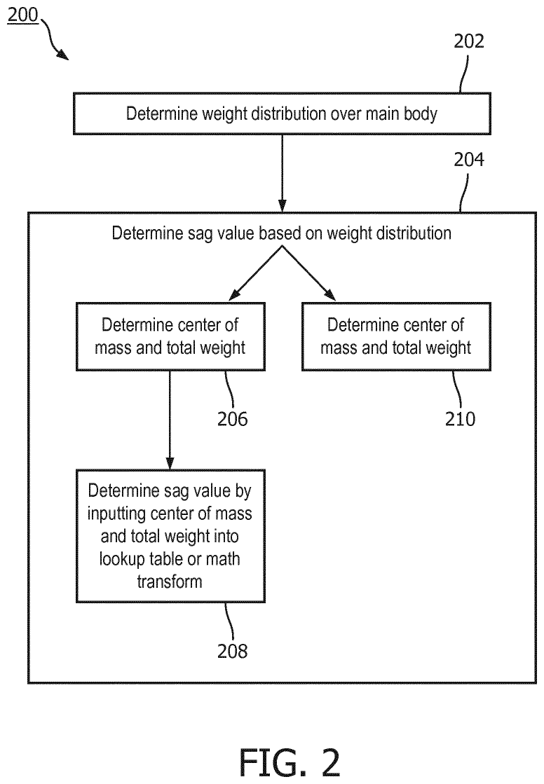

With reference to FIG. 2, the sag estimation operation 200 is diagrammatically shown as a flowchart. At 202, a weight distribution is determined over the top surface 14 of the main body 12 based on readings of the pressure sensors 16. At 204, a sag value of the main body 12 is determined based on the weight distribution. To do so, in one example at 206, a center of mass and a total weight are determined for the weight distribution. At 208, a sag value is determined by inputting the center of mass and total weight values to a look-up table or mathematical transform (e.g., stored on the non-transitory storage medium read by the computer 20). In another example, at 210, the sag value is determined by integrating or summing sag contributions of weight portions of the patient's body over the weight distribution. Once the sag value is estimated, the sag value can be used to correct the imaging data for the positon of the patient on the top surface 14 of the main body 12 during the imaging procedure. In another contemplated embodiment, no such sag correction is performed, but instead an excessive sag warning is output, e.g. on the display of the computer 20, if the sag exceeds some chosen alarm threshold.

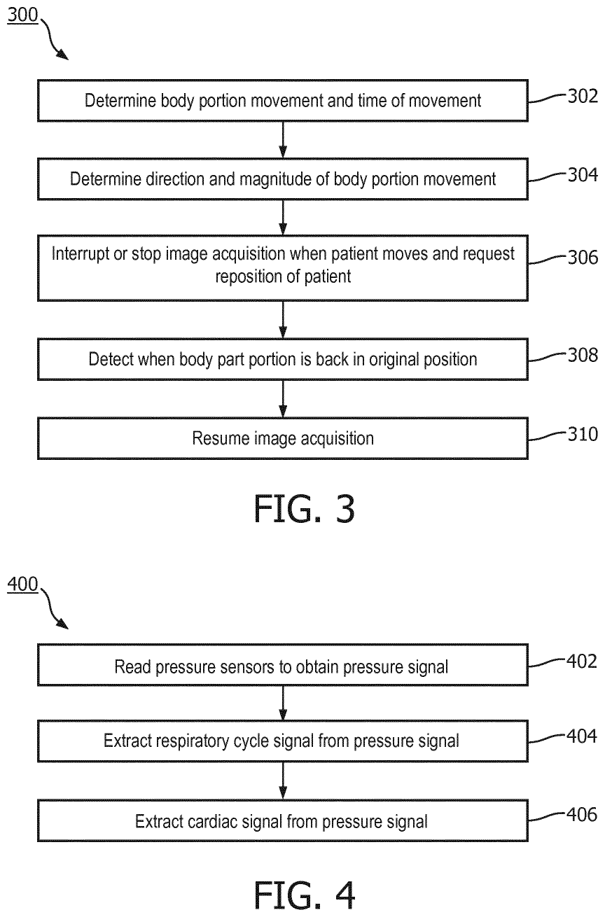

With reference to FIG. 3, the motion estimation operation 300 is diagrammatically shown as a flowchart. This motion estimation 300 may be usefully performed, for example, during execution of the imaging data acquisition process 100 in order to detect volitional patient motion and optionally remediate such motion if appropriate. At 302, a portion of the patient's body that moves on the top surface 14 during the imaging procedure is identified, and a time that the portion of the patient's body moves is determined. At an optional operation 304, a direction and magnitude of the portion that the patient's body moves is determined. At 306, the imaging data acquisition process 100 performed by the scanner 18 under control of the electronic processor 22 is interrupted or stopped from obtaining images of the patient, and a request to reposition the portion of the patient's body that moved back into its original position is issued, e.g. by displaying on the display of the computer 20. At 308, the processor 22 is programmed to continually (or at rapid intervals) read the pressure sensors 16 to detect when the portion of the patient's body that moved is repositioned in its original position. To do so, the pressure distribution recorded prior to the motion detection event 302 is compared with the pressure distribution currently being read, and when these agree to within a chosen tolerance the patient is deemed to have moved the body part back to its original position. In some embodiments, further prompts may be issued—for example, if it is detected that the body part has moved close to its original position but is still (for example) five centimeters offset to the right of its original position, then a further prompt may be issued requesting that the patient move the body part (e.g. leg, or arm) another five centimeters to the left. At 310, once the processor 22 detects that the portion of the patient's body is repositioned, the image data acquisition is resumed by the scanner 18.

In a variant embodiment, the remediation is performed by considering the impact of the moved body part in context of the imaging data acquisition process 100. In this embodiment, the time of the movement determined at operation 302 is compared with the state of progress of the imaging data acquisition process 100. In the case of an acquisition such as a whole body scan, it is typical for the imaging to progress sequentially from head to foot either continuously or in a certain number of steps. In such a case, if the moved body part has already been imaged then the movement is not of consequence, and no action is taken. On the other hand, if the moved body part has not yet been scanned or needs to be additionally scanned, then some remediation is called for. This may involve the process of FIG. 3 by which the patient is instructed to move the body part back to its original position. In another remedial approach, if the direction and distance of body part movement is determined in the operation 304 (e.g. by comparing the weight distributions acquired before and after the movement is detected in operation 302) then the imaging data acquired before and after the movement detected in operation 302 may be separately reconstructed, and the two resulting images may then be spatially registered using the movement direction and distance information from operation 304 as initial values for the spatial registration adjustment.

In another contemplated remedial approach, if the detection of movement 302 occurs early in a data acquisition then the imaging data acquired prior to the movement may be discarded. Optionally, the data acquisition process 100 may also be extended in time to compensate for the loss of the discarded imaging data. In yet another contemplated remedial approach, the detection of movement 302 may cause the data acquisition process 100 to be aborted entirely and repeated, optionally with a message issued cautioning the patient to remain still during the imaging data acquisition process 100.

It is also contemplated for the instructions stored on the non-transitory storage medium to include instructions for carrying out any chosen one of these options and a decision may be made based on the time of the movement detected in the operation 302 in the context of the ongoing imaging data acquisition process 100. For example, if the movement is detected less than some threshold time into the data acquisition process 100 then the approach of discarding the early data may be employed; whereas if the movement is detected after passing that threshold time into the data acquisition process 100 then another remedial approach may be taken such as aborting and repeating the acquisition process 100 in its entirety, or inducing the patient to reposition the moved body part as per the process flow charted in FIG. 3.

The choice of which remedial action to take may also optionally depend on the criticality of the moved body part for example, the movement of a foot during a torso scan may be of little relevance (so that no remediation is performed); whereas, the movement of a lower arm during such a torso scan is likely to have a small effect that can be corrected by inducing repositioning of the lower arm as per the approach of FIG. 3; whereas, movement of the shoulder is likely to have a large effect on the torso scan and may require the most invasive remediation of aborting the torso scan and repeating it.

With reference to FIG. 4, the respiratory monitoring operation 400 is diagrammatically shown as a flowchart. Again, this process 400 is preferably performed concurrently as the imaging data acquisition process 100 executes. At 402, the pressure sensors 16 that contact a portion of the patient's body on the top surface 14 of the main body 12 are read to obtain a pressure magnitude versus time signal. At 404, a respiratory cycle signal is extracted from the pressure magnitude versus time signal. This may entail, for example, filtering the pressure magnitude versus time signal to extract the component at the breathing frequency. At 406, a cardiac cycle signal is optionally extracted from the pressure magnitude versus time signal, e.g. by filtering to extract the signal component at the heart rate frequency. The respiratory signal-versus-time is preferably recorded, and may be used to perform respiratory gating of the imaging data acquired by the concurrently executing imaging data acquisition process 100. Such gating may be done retrospectively, e.g. by time-stamping the imaging data (e.g. individual counts in emission imaging) as it is acquired and then binning the imaging data into respiratory phase bins based on the respiratory phases indicated by the respiratory signal. Alternatively, in a prospective respiratory gating process, the imaging data acquisition process 100 is prospectively controlled to acquire imaging data only when the patient's breath cycle is in the chosen respiratory phase.

The effectiveness of the respiratory monitoring process 400 of FIG. 4 depends on how well the pressure magnitude reflects the respiration. This correlation is expected to be strongest for those pressure sensors that contact the torso of the patient. Accordingly, in some embodiments the pressure read operation 402 reads only those pressure sensors 16 in the vicinity of the torso. Additionally, in the operation 404 it is contemplated to perform a selection process to extract the respiratory signal from the pressure sensor 16 whose pressure magnitude signal most strongly correlates with respiration (or, to extract the respiratory signal from a small group of pressure sensors whose pressure magnitude signals most strongly correlates with respiration). This may be done, for example, by transforming the pressure magnitude versus time signal into the frequency domain, e.g. using a Fourier transform, and ranking the pressure sensors 16 by signal strength in the frequency band corresponding to credible breathing rates (e.g., an adult at rest draws typically about 12-20 breaths per minute, so the frequency band of credible breathing rates may be in the range of 8-24 cycles/minute).

Similar processing may be performed for the operation 406 to improve detection of the cardiac cycling signal. Again, pressure sensors in the vicinity of the torso are expected to provide the strongest cardiac cycling signal, and sensor ranking in this case may be by signal strength in the credible heart rate band, e.g. on the order of 40-150 cycles/minute corresponding to the credible range of heart rate for a typical adult.

The disclosure has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A device for a patient to lie on during a medical imaging procedure, the device comprising:
a main body;
a matrix of pressure sensors disposed on a top surface of the main body, the pressure sensors being configured to measure pressure across the top surface;
at least one electronic processor operatively connected to read the pressure sensors; and
a non-transitory storage medium storing instructions readable and executable by the at least one electronic processor to use the matrix of pressure sensors to perform at least one of:
a sag estimation operation;
a motion estimation operation; and
a respiratory monitoring operation.

2. The device of claim 1, wherein the non-transitory storage medium stores instructions readable and executable by the at least one electronic processor to perform a sag correction estimation operation comprising:
determining a weight distribution over the top surface of the main body based on the readings of the pressure sensors; and
determining a sag value quantifying sag of the main body based on the weight distribution.

3. The device of claim 2, wherein the sag estimation operation further includes:
determining a center of mass and a total weight of the weight distribution over the top surface of the main body; and
determining the sag value by inputting the center of mass and the total weight to a look-up table or mathematical transform.

4. The device of claim 2, wherein the sag estimation operation further includes:
determining the sag value by integrating or summing sag contributions of portions of the weight distribution over the weight distribution.

5. The device of claim 1, wherein the non-transitory storage medium stores instructions readable and executable by the at least one electronic processor to perform a motion estimation operation including:
using the matrix of pressure sensors, determining a portion of the patient's body that moves from an original position and a time that the portion of the patient's body moves from its original position, wherein the motion estimation operation further includes:
determining a direction and magnitude that the portion of the patient's body moves.

6. (canceled)

7. The device of claim 5, wherein the motion estimation operation further includes:
interrupting an imaging data acquisition and generating a request to reposition the portion of the patient's body that moved back in its original position;
using the matrix of pressure sensors, detecting when the portion of the patient's body that moved is repositioned in its original position; and
resuming the imaging data acquisition after the detecting wherein, when movement of a portion of the patient's body is detected, the at least one electronic processor is further programmed to perform at least one remedial operation selected from:
generating an instruction for the patient to move the moved body portion back to its original position;
separately reconstructing images acquired before and after the movement is detected;
discarding images acquired prior to the detection of the movement; and
generating an instruction to restart acquiring the images.

8. (canceled)

9. The device of claim 7, wherein the non-transitory storage medium stores instructions readable and executable by the at least one electronic processor to perform a respiratory monitoring operation including:
reading the pressure sensors that contact a portion of the patient's body on the top surface of the main body to obtain a pressure magnitude versus time signal, and
extracting a respiratory cycle signal from the pressure magnitude versus time signal.

10. The device of claim 9, wherein the non-transitory storage medium further stores instructions readable and executable by the at least one electronic processor to perform a cardiac monitoring operation including:
extracting a cardiac cycle signal from the pressure magnitude versus time signal.

11. (canceled)

12. A device for a patient to lie on during a medical imaging procedure, the device comprising:
an imaging device;
a main body arranged to load a patient into the imaging device for imaging;
a matrix of pressure sensors disposed on a top surface of the patient support, the pressure sensors being configured to measure pressure across the top surface;
at least one electronic processor operatively connected to read the pressure sensors; and
a non-transitory storage medium storing instructions readable and executable by the at least one electronic processor to use the matrix of pressure sensors to perform at least one of:
a sag estimation operation;
a motion estimation operation; and
a respiratory monitoring operation.

13. The device of claim 12, wherein the sag estimation operation includes:
determining a weight distribution over the top surface of the main body based on the readings of the pressure sensors;
determining a center of mass and a total weight of the weight distribution; and
determining a sag value quantifying sag of the main body by inputting the center of mass and the total weight to a look-up table or mathematical transform.

14. The device of claim 12, wherein the sag correction operation includes:
determining a weight distribution over the top surface of the main body based on the readings of the pressure sensors; and
determining the sag value by integrating or summing sag contributions of portions of the weight distribution over the weight distribution.

15. The device of claim 12, wherein the motion estimation operation includes:
using the matrix of pressure sensors, determining a portion of the patient's body that moves from an original position and a time that the portion of the patient's body moves from its original position.

16. The device of claim 15, wherein the non-transitory storage medium further stores instructions readable and executable by the at least one electronic processor to control the imaging device to perform an imaging data acquisition process, and the motion assessment operation further includes:
stopping the imaging data acquisition process in response to determining the portion of the patient's body has moved from its original position;
generating a request to reposition the portion of the patient's body that moved during the image acquisition back in its original position;
using the matrix of pressure sensors, detecting when the portion of the patient's body that moved is repositioned in its original position; and
resuming the imaging data acquisition process after the detecting that the patient's body that moved is repositioned in its original position.

17. The device of claim 15, wherein the non-transitory storage medium further stores instructions readable and executable by the at least one electronic processor to:
control the imaging device to perform an imaging data acquisition process;
determine based on the portion of the patient's body that moves from its original position and the time that the portion of the patient's body moves from its original position whether the imaging data acquisition process has already acquired imaging data for the portion of the patient's body that moves at the time that the portion of the patient's body moves; and
interrupt or stop the imaging data acquisition process only if the imaging data acquisition process has not already acquired imaging data for the portion of the patient's body that moves at the time that the portion of the patient's body moves.

18. The device of claim 12, wherein the respiratory monitoring operation includes:
reading the pressure sensors that contact a portion of the patient's body on the top surface of the main body to obtain a pressure magnitude versus time signal, and
extracting a respiratory cycle signal from the pressure magnitude versus time signal.

19. A method of monitoring a patient during an image acquisition procedure, the method including:
reading pressure sensors that contact a portion of the patient's body on a top surface of a main body to obtain pressure data; and
based on the obtained pressure data, performing at least one of:
estimating a sag of the main body;
estimating motion of a portion of the patient's body; and
monitoring respiration of the patient.

20. The method of claim 19, wherein the sag of the main body is estimated, and the estimating of the sag includes:
determining a weight distribution over the top surface of the main body from the pressure data; and
determining the sag of the main body based on the weight distribution by:
determining a center of mass and a total weight of the weight distribution; and
inputting the center of mass and the total weight to a look-up table or mathematical transform that outputs the sag.

21. The method of claim 19, wherein the sag of the main body is estimated, and the estimating of the sag includes:
integrating or summing sag contributions of weight portions over the weight distribution.

22. The method of claim 19, wherein motion of a portion of the patient's body is estimated, and the estimating of the motion includes:
stopping an imaging data acquisition process in response to determining the portion of the patient's body has moved from its original position;
generating a request to reposition the portion of the patient's body that moved during the image acquisition back in its original position;
using the pressure sensors, detecting when the portion of the patient's body that moved is repositioned in its original position; and
resuming the imaging data acquisition process after the detecting that the patient's body that moved is repositioned in its original position.

23. The method of claim 19, wherein monitoring respiration of the patient includes:
reading the pressure sensors that contact a portion of the patient's body on the top surface of the main body to obtain a pressure magnitude versus time signal, and
extracting a respiratory cycle signal from the pressure magnitude versus time signal.
